(19) United States
(12) Patent Application Publication
(10) Pub. No.: US 2018/0114596 A1
(43) Pub. Date: Apr. 26, 2018

(54) SYSTEMS AND METHODS FOR GENERATING CUSTOM USER EXPERIENCES BASED ON HEALTH AND OCCUPATIONAL DATA

(71) Applicant: Vimedicus, Inc., Chicago, IL (US)

(72) Inventors: Thomas Churchwell, Chicago, IL (US); Robert Churchwell, Chicago, IL (US); Stephen Wasko, Chicago, IL (US); Katherine Francis, Chicago, IL (US); Kellie Schoen, Chicago, IL (US); Corey Campbell, Chicago, IL (US); Sally Rosenthal, Chicago, IL (US); Willibald G. Berlinger, Peoria, IL (US)

(21) Appl. No.: 15/847,714

(22) Filed: Dec. 19, 2017

Related U.S. Application Data
(63) Continuation-in-part of application No. 15/686,734, filed on Aug. 25, 2017.
(60) Provisional application No. 62/379,256, filed on Aug. 25, 2016.
(60) Provisional application No. 62/436,162, filed on Dec. 19, 2016.

(51) Int. Cl. G16H 10/60; G16H 20/00; G16H 40/63

(52) U.S. Cl. CPC G16H 10/60; G16H 40/63; G16H 20/00

(57) ABSTRACT

A system obtains health data corresponding to a plurality of patients from one or more first data sources and occupational data from one or more second data sources. The system processes the health data and the occupational data to identify at least one patient of a plurality of patients for inclusion in a behavioral modification program for a health condition of the patient. The system also generates graphical user interfaces of the behavioral modification program for display at a client device to display content associated with the health condition. The system monitors user interactions with the content and integrates additional content based on the monitored user interactions.

### CROSS-REFERENCE TO RELATED APPLICATION

This application is a continuation-in-part of U.S. patent application Ser. No. 15/686,734, filed Aug. 25, 2017, and titled “Systems and Methods for Generating Custom User Experiences Based on Processed Claims Data” which claims the benefit of U.S. Provisional Patent Application No. 62/379,256, filed Aug. 25, 2016, entitled “System and Method for Health Improvement”. This application further claims the benefit of U.S. provisional application No. 62/436,162 titled “System and Method for Health Improvement”, filed Dec. 19, 2017. Each of these applications is hereby incorporated by reference in their entirety.

### FIELD

Aspects of the present disclosure relate to computing devices and hardware used in data aggregation and processing of health and occupational data to generate and provide customized patient plans.

### BACKGROUND

Developing and implementing a treatment or care plan for a patient often requires tailoring the specific objectives and approaches of the plan to the patient's particular injury or illness. Moreover, successful completion of a treatment or care plan is also heavily dependent on the plan aligning with the patient's personality, goals, and lifestyle.

The selection or offering of a treatment plan to a patient may be based on a wide range of health-related data. For example, healthcare providers (such as hospitals, clinics or physicians) typically send claims to healthcare payer institutions to obtain reimbursement for services rendered to a patient. Typically, the claims are sorted, indexed and electronically stored. As a result, such claims data may be used to identify and suggest suitable treatment plans for a patient. Employers similarly track and store occupational data regarding their employees. Such data may include the number of days an employee is absent or unable to work at their peak performance due to injury or illness.

Accordingly, there is a need for treatment plan systems that account for a patient's unique characteristics in developing and implementing a plan and, once implemented, that modify the plan in order to accommodate changes in the patient. There is a further need for such a system that identifies and prioritizes specific patients or employees for enrollment in treatment plans to maximize the efficiency with which health care benefits are applied.

### SUMMARY

In one implementation of the present disclosure, a system is provided that includes a processing device and a memory containing instructions that, when executed by the processing device, cause the processing device to obtain health data corresponding to a plurality of patients from one or more first data sources. The instructions further cause the processing device to obtain occupational data from one or more second data sources, the occupational data corresponding to the plurality of patients and to process the health data and the occupational data to identify at least one patient of the plurality of patients for inclusion in a behavioral modification program for at least one health condition corresponding to the patient. The instructions also cause the processing device to generate one or more graphical user interfaces of the behavioral modification program for display at a client device, the one or more graphical user interfaces displaying content associated with the health condition, and to monitor user interactions with the first content. The instructions further cause the processing device to integrate additional content into the graphical user interfaces based on the monitored user interactions with the content.

In another implementation of the present disclosure, a non-transitory computer readable medium encoded with instructions executable by a computing device is provided. When executed by the computing device, the instructions cause the computing device to obtain health data corresponding to a plurality of patients from one or more first data sources. The instructions further cause the computing device to obtain occupational data from one or more second data sources, the occupational data corresponding to the plurality of patients and to process the health data and the occupational data to identify at least one patient of the plurality of patients for inclusion in a behavioral modification program for at least one health condition corresponding to the patient. The instructions also cause the computing device to generate one or more graphical user interfaces of the behavioral modification program for display at a client device, the one or more graphical user interfaces displaying content associated with the health condition, and to monitor user interactions with the first content. The instructions further cause the computing device to integrate additional content into the graphical user interfaces based on the monitored user interactions with the content.

### BRIEF DESCRIPTION OF THE FIGURES

The foregoing and other objects, features, and advantages of the present disclosure set forth herein will be apparent from the following description of particular embodiments of those inventive concepts, as illustrated in the accompanying drawings. Also, in the drawings the like reference characters refer to the same parts throughout the different views. The drawings depict only typical embodiments of the present disclosure and, therefore, are not to be considered limiting in scope.

FIG. 1 is a block diagram illustrating a computing architecture for processing health and occupational data to automatically provide content to patients identified for inclusion in a behavioral modification plan according to aspects of the present disclosure.

FIG. 2 is a flowchart of an example process for processing health and occupational data according to aspects of the present disclosure.

FIG. 3 is an example claims data record, according to aspects of the present disclosure.

FIG. 4 is an example occupational data record according to aspects of the present disclosure.

FIG. 5 illustrates an example health data source according to aspects of the present disclosure.

FIG. 6 illustrates an example combined health and occupational data source according to aspects of the present disclosure.

FIG. 7 is a block diagram illustrating a computing device according to aspects of the present disclosure.

### DETAILED DESCRIPTION

The healthcare ecosystem—from patients and providers to payers and product developers—has seen ever-growing costs, significantly associated with patients that suffer from multiple chronic medication conditions and illnesses. For example, well-known chronic illnesses include chronic obstructive pulmonary disorder (COPD), congestive heart failure (CHF), obesity, hypertension, chronic pain, asthma, and diabetes. While advances in medicine have improved the life span for patients suffering from such chronic conditions, the medical industry has struggled to reverse the progression of these diseases once a patient has been diagnosed. Furthermore, patients who suffer from one chronic disease often develop additional chronic illnesses. Such a downward cascade can be debilitating to the patient, taxing for providers, and expensive for payers.

In an attempt to resolve such issues, many healthcare related computing systems have been created that attempt to aid patients in their treatment of chronic illnesses and conditions. For example, typical systems involve an application for a mobile device, such as a smart phone having a processor and wireless networking capability, which allows users to manage and track treatments of a chronic disease. Further, such systems manage and track treatments of chronic diseases one disease at a time rather than addressing the conditions that lead to the development of multiple co-morbid chronic diseases. Such systems are data-driven and require users (i.e., patients) to manually input vast amounts of information related to their individual chronic conditions. Also, such systems typically recommend disease-specific treatment services to users rather than more broad-based behaviorally oriented services that are needed to treat users with multiple chronic diseases. To discern the appropriate multiple chronic disease patterns and recommend an appropriate treatment, such systems require a vast amount of disparate data that must often be manually entered.

Moreover, existing systems are unable to process and incorporate medical claims data and other clinical health data that provide meaningful insight into the constellation of a patient's multiple chronic conditions and overall medical condition. For example, the majority of healthcare providers (physicians, dentists, etc.) obtain payment for medical services provided to a patient from a payer, which is generally a healthcare organization or insurance company administering a plan for the patient's employer. The data that is submitted from the healthcare provider to the payer is generally referred to as a “claim” and includes the information used by a payer for payment of the healthcare provider for the services rendered to the patient. In one specific example, the claim represents a set of data, or an electronic document that may include, among other things, an identification code for the physician that provided the service, one or more diagnostic codes, a service identification code, an identifier corresponding to the patient, the patient's group and plan number, a payer identifier, the amount of the claim, and a co-payment amount, all of which may be maintained in a database, or other type of data structure.

Typical health care computing systems are unable to process claims data for several reasons including, without limitation, the vast amount of information and associated records involved, the existence of disparate and/or inconsistent data sets, and the general complexity of accessing claims computing data from multiple claims data storage systems. Claims computing systems often process a high volume of claims in accordance with dynamic medical policies but do not have the ability to completely and accurately process such claims data sets. Moreover, identifying trends within a complete set of claims and medical data can be cumbersome.

Claims data also provides only a partial understanding of a patient's health based on the products and procedures procured by the patient and, as a result, may lead to an incomplete or otherwise suboptimal treatment plan. A more sophisticated and complete treatment plan generally requires an understanding of a patient's day-to-day activities, which often coincide with a patient's job.

Thus, a need exists for a more efficient, complete and cost-effective system for generating and managing health-related data to generate interactive patient treatment plans that further incorporate broader aspects of a patient's activities, such as the patient's work-related activities and performance.

Aspects of the present disclosure solve these specific technical issues by providing a system and method that automatically processes large amounts of health and occupational data in real-time and identifies patients as potential participants for inclusion into a behavior modification treatment program directed to addressing health conditions of the patient. The systems and methods further provide for ongoing management and modification of the behavioral modification program based on feedback provided by various sources, such as the patient, the patient's physician, the patient's employer, and the like. The health and occupational data used to identify and develop a given behavioral modification program may include, without limitation, one or more of claims data associated with the patient, medical records associated with the patient, physical activity of the patient, and employment-related data of the patient. For purposes of this disclosure, the terms “patient”, “employee”, and “patient/employee” are generally interchangeable and are used to denote individuals for which health and occupational data from which a suitable behavioral modification program may be identified is available.

In certain implementations, for example, the health and occupational data may be stored in one or more tables or similar data structure that include various fields and/or parameters that are processed by the system in conjunction with one or more rules or algorithms that enable the system to identify specific lifestyle change needs and chronic conditions of a patient. Once a lifestyle change need for a given patient has been verified, the patient may be flagged as a potential participant for a behavioral modification program, and the system may provide (e.g., via graphical user interface) customized content (e.g., multimedia content) to the patient that targets the specific aspects of the participants chronic conditions and/or behavior, thereby offering more meaningful and engaging treatment to the patient. Such content may include, without limitation, one or more of surveys, questionnaires, articles, images, videos, games, tracking sheets, graphs, tables, websites, infographics, and any other content directed to providing the patient with information regarding their health or information regarding the patient's progress through a behavioral modification program in which the patient is enrolled. Since the content is targeted specifically to the patient's intrinsic needs, the patient is more likely to engage and interact with the content and thereby actively participate in the behavioral modification program.

Further, once a patient is flagged as a potential participant for a behavioral modification program, the system may provide (e.g., via graphical user interface) recommended treatment curriculum (e.g., multimedia content) to a coach or a guide for the patient that is specific to aspects of the patient's chronic conditions and/or behavior, thereby allowing the coach or similar healthcare professional to deliver more meaningful and better treatment to the patient. Since the content is targeted specifically to the patient's intrinsic needs, the content will be better tailored by the coach to the specifically identified patient.

In other aspects, the disclosed system may include a mechanism that continuously monitors a patient's interactions with the provided content. In one specific example, the system may monitor patient interactions with the content and/or graphical user interfaces in order to identify decisions made by the patient with respect to the patient's engagement in the behavioral modification program. Data corresponding to such interactions may be used to monitor the patient's progress and to present actual behavior-based information to the patient or a healthcare professional working with the patient for continuous treatment modification, thereby contributing to greater success in achieving health goals.

The term “behavioral modification program”, as used in this disclosure, generally refers to a dynamically generated collection of content directed to treatment of one or health conditions of an identified patient. As described in further detail in this specification, the particular content included in a behavioral modification program may vary in both form and substance depending on the particular health conditions of the patient and goals provided by one or more of the patient, the patient's employer, or a healthcare professional working with the patient. In general, however, the purpose of the behavioral modification program is to provide recommendations and guidance to a patient to facilitate recovery or management of one or more health conditions and the behavioral modification program may be directed to any aspects aimed at achieving such a purpose. For example, some aspects of the behavioral modification program may be specifically directed to changing or recommending changes to a patient's behavior. Such behavior may include elements of the patient's physical activity, sleep, diet, or similar activities in which the patient engages. Other aspects of the behavioral modification program may instead be directed to collecting information from the patient for purposes of tracking the patient's progress. Such content may include questionnaires, surveys, or other content for collecting data regarding, among other things, the physical condition and experiences of the patient. Certain aspects of the behavioral modification program may be directed primarily to providing encouragement, feedback, and motivation to the patient, such as motivational articles or videos.

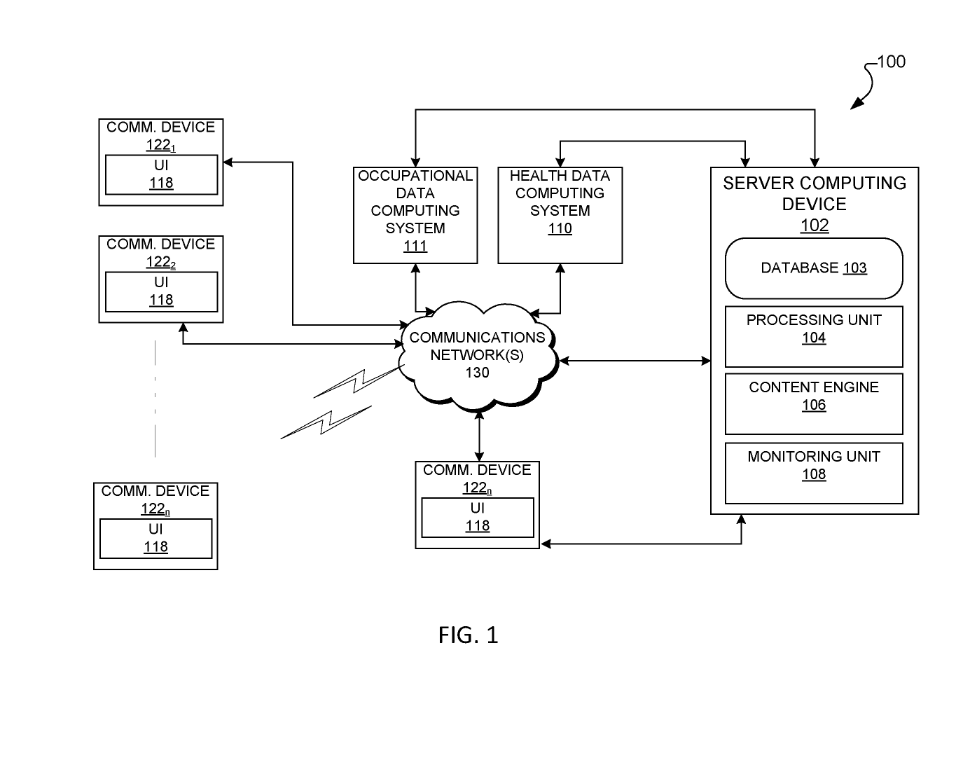

FIG. 1 provides an illustrative example of a computing network 100 that may be used to process health and occupational data to identify participants for a behavioral modification program, according to one embodiment. As illustrated, the computing network 100 includes various devices functioning together in the gathering and processing of health and occupational data. In the illustrated embodiment, the computing network 100 includes server computing device 102 that includes a processing unit 104 for processing health and occupational data to, among other things, identify participants for inclusion in a behavioral modification program and automatically identifying custom content for initial inclusion in the behavioral modification program. The server computing device 102 further includes a database and/or data store 103 (or some other database architecture including those embodied in a single database or multiple databases of the same or differing platforms) that is used to store, among other information and content, user health-related data, data relating to healthcare content, health-care related applications, data generated from users interacting with graphical user-interfaces generated by the server computing device 102, and user occupational data.

Health and occupational data that may be collected, stored, and processed by the server computing device 102 may originate from various sources. In some implementations of the present disclosure, for example, health and health-related data may include claims data obtained from a claims data computing system, such as a claims data computing system located at a health care provider, hospital, physician's office, or other entity involved in processing and maintaining claims data. In such implementations, the server computing device 102 functionally communicates with a health data computing system 110. Such health related data may be stored in one or more formats and across multiple data sources. Examples of formats in which the health data may be stored include, without limitation, formats conforming to one or more of the Health Level Seven (HL7) standard, the Systematized Nomenclature of Medicine (SNOMED) standard, and the Logical Observation Identifiers Names and Codes (LOINC) standard.

Occupational data may include work-related data associated with employees of an organization. In some implementations of the present disclosure, for example, the occupational data may be obtained from one or more occupational data computing systems, such as occupational data computing system 111. The occupational data computing system 111 is generally a computing system that stores and maintains records associated with workers and work-related tasks for one or more employers, companies, or similar organizations. In certain implementations, the occupational data computing system 111 may be a human resources information system (HRIS) or similar information system that stores and maintains information regarding organization personal. Accordingly, the occupational data may include actual data associated with a specific worker. For example, occupational data associated with a particular worker may include, without limitation, general measures of worker productivity, absenteeism data (i.e., data indicating instances in which a worker failed to report to work), and presenteeism data (i.e., data indicating instances in which a worker reported for work but was ill or otherwise unable to perform at peak levels). Occupational data may also include data associated with a worker's job description or tasks to be performed by the worker. For example, occupational data may include descriptions of tasks to be performed by the worker, physical demands or requirements associated with such tasks (e.g., max lifting loads), and health-related conditions that may preclude or otherwise render a worker unable to perform such tasks efficiently and safely. The occupational data may also include such elements as production throughput per time unit, average hours worked per shift, supervisor quantitative performance evaluation data, and whether the employee is on a Performance Improvement Plan (PIP).

The process of collecting and analyzing data from each of the health data computing system 110 and the occupational data computing system 111 may include normalizing data from one or both of the computing systems 110, 111 into a consistent format. Collecting and analyzing data from the computing systems 110, 111 may also include summarizing, generating one or more algorithm results, or otherwise determining one or more derived pieces of data from the data stored in the computing systems 110, 111.

The server computing device 102 includes a content engine 106 that uses the processed health and occupational data to identify participants for inclusion into a behavioral health care program or similar behavioral modification program. Additionally, based on the identified participant, the content engine 106 may identify specific content for inclusion in the behavioral modification program such that the content is presented to the identified participant.

The server computing device 102 further includes a monitoring unit 108 that continuously monitors an identifier patient's interaction with the content provided by the content engine 106. In some embodiments, the monitoring engine 106 may generate one or more graphical user interfaces that, as will be described in more detail below, generate various metrics and graphical displays articulating the patient's interactions with the provided content.

One or more communication devices 122₁, 122₂,-122_{N}, functionally communicate with the server computing device 102 using a communications network 130. Accordingly, the one or more communication devices 122₁, 122₂,-122_{N} may include, without limitation, one or more of a personal computer, a work station, a mobile device, a mobile phone, a tablet device, or any other type of network-enabled processing device capable of implementing and/or executing processes, software, applications, etc. Each of the one or more communication devices 122₁, 122₂,-122_{N} is further capable of executing software or other instructions for presenting a user interface 118 to a user of one of the communication devices 122₁, 122₂,-122_{N}. The user interface 118 generally enables presentation of information received from the server computing device 102 and receipt of commands from the user to control or otherwise interact with software or applications executed by the communication devices 122₁, 122₂,-122_{N}. In certain instances, such interaction may include receiving commands from a user at one of the communication devices 122₁, 122₂,-122_{N} and transmitting the received commands to the server computing device 102 over the communications network 130. Additionally, the one or more communication devices 122₁, 122₂,-122_{N}, may include one or more processors that process software or other machine-readable instructions and may include a memory to store the software or other machine-readable instructions and data.

The communications network 130 may include via or more wireless networks such as, but not limited to one or more of a Local Area Network (LAN), Wireless Local Area Network (WLAN), a Personal Area Network (PAN), Campus Area Network (CAN), a Metropolitan Area Network (MAN), a Wide Area Network (WAN), a Wireless Wide Area Network (WWAN), Global System for Mobile Communications (GSM), Personal Communications Service (PCS), Digital Advanced Mobile Phone Service (D-Amps), Bluetooth, Wi-Fi, Fixed Wireless Data, 2G, 2.5G, 3G, 4G, LTE networks, enhanced data rates for GSM evolution (EDGE), General packet radio service (GPRS), enhanced GPRS, messaging protocols such as, TCP/IP, SMS, MMS, extensible messaging and presence protocol (XMPP), real time messaging protocol (RTMP), instant messaging and presence protocol (IMPP), instant messaging, USSD, IRC, or any other wireless data networks or messaging protocols. Network 130 may also include wired networks.

Although described above as primarily corresponding to a patient, one or more of the communication devices 122₁, 122₂,-122_{N}, may instead correspond to a healthcare professional (such as, without limitation, a physician, an occupational health nurse, a physical therapist, an occupational therapist, etc.) and may allow access by the healthcare professional to one or more aspects of the server computing device 102. Such access may enable the healthcare professional to, among other things, monitor a patient's progress through the patient's behavioral modification program; add, delete, modify or otherwise change aspects of a particular patient's behavioral modification program; modify existing aspects of a patient's behavioral modification program; exchange communication with the patient; and generate new content that may be provided to patient's as part of a behavioral modification program.

As previously noted, the server computing system 102 generally obtains data from data sources associated with the health data computing system 110 and the occupational data computing system 111. The server computing system 102 may also be configured to retrieve additional data from one or more other computing systems. Such computing systems may include, without limitation, one or more electronic medical record (EMR) systems or similar systems for storing and maintaining patient medical records. Such systems may, for example, be associated with a hospital, a clinic, or similar medical facility.

The server computing system 102 may also be adapted to receive additional data regarding the patient from the patient and/or one or more other individuals, such as healthcare professionals involved in the patient's treatment. For example, in certain implementations, the patient and/or healthcare professional may add, delete, modify, or otherwise change data corresponding to the patient that is stored or accessible by the server computing system 102. Such changes may be made, for example, using one of the communication devices 122₁, 122₂,-122_{N}.

Data obtained and processed by the server computing system 102 may also include social media data associated with a patient. For example, in certain implementations, the server computing system 102 may access publicly available social media information to identify relevant interests and group memberships of patients. Such information may include a patient's group and forum memberships, participation in such groups and forums, “likes” or similar comments regarding particular content, and the like. For example, social media data may include, among other things, a patient's membership in groups or forums associated with one or more of particular health conditions, particular wellness content providers, and the patient's occupation.

In certain implementations, the server computing system 102 may be configured to generate and transmit automated reminders, messages, alerts, or other communications to one or more of the communication devices 122₁, 122₂,-122_{N}. Such communications may include, without limitation, one or more of an email, a text message, an automated phone call, an in-application alert or message, a notification, or any other suitable form of communication. In certain implementations, the automated communications may be generated and transmitted in response to certain conditions. Such conditions may include, without limitation, a patient failing to complete (in whole or in part) an aspect of their treatment plan (such as a piece of content included in the treatment plan), a patient completing (in whole or in part) an aspect of their treatment plan, a certain time elapsed since an aspect of a treatment plan was initiated or completed, and the like. In certain implementations, automated communications may also be used to remind a patient to, among other things, take certain medication, perform certain exercises, or otherwise perform some schedule activity.

The server computing system 102 may further enable communication between a patient and a healthcare professional or other individual tasked with guiding the patient through, among other things, the system and/or the patient's specific behavioral modification program. Such communication may generally occur between two or more of the communication devices 122₁, 122₂,-122_{N}, or any of the communication devices 122₁, 122₂,-122_{N} and one or more other computing devices communicatively coupled to the communications network 130. Communication facilitated by the server computing system 102 may include, without limitation, one or more of online messages, text messages, phone calls (including conference calls with multiple participants), e-mails, videoconferences, and any other suitable form of communication. In light of the foregoing, the patient and any healthcare professionals and/or other individuals tasked with monitoring the patient's completion of his or her behavioral modification program may be remotely located form the patient.

In certain implementations, involvement of healthcare professionals or other individuals in administering a particular behavioral modification program may require explicit approval or permission from the patient. For example, after the server computing system 102 identifies a particular patient for enrollment in a behavioral modification program and generates the behavioral modification program, details of the behavioral modification program may be presented to the patient. The patient may then review the behavioral modification program to determine whether the behavioral modification program suits his or her particular goals, lifestyle, etc. If the patient disapproves of one or more aspects of the behavioral modification program, the patient may be presented with additional questionnaires, surveys, or other feedback mechanisms to further refine the behavioral modification program to the patient's specific needs, objectives, or personal preferences. If, on the other hand, the patient approves the behavioral modification program, the patient may be presented with an electronic contract or other commitment regarding the patient's participation in the behavioral modification program. The patient may also be asked to identify or otherwise confirm and approve one or more individuals who may have access to some or all of the patient's data during administration of the behavioral modification program.

Although previously described as obtaining health and occupational data during the process of identifying patients and generating corresponding behavioral modification programs, the server computing system 102 may also retrieve such data during the course of a patient's behavioral modification program. In response to retrieving updated data pertaining to a patient, the system 102 may perform an additional analysis of the updated data to identify potential modifications to the patient's existing behavioral modification program. Such changes may include, without limitation, addition, deletion, or modification of content assigned to the behavioral modification program and changes to particular aspects of the behavioral modification program such as, without limitation, the duration, intensity, or type of physical therapy activities assigned to the patient or the quantity, type, or frequency of medication, food, or other items consumed or taken by the patient. A patient's health and/or occupational data may also be periodically analyzed even if no update to that data has occurred. By doing so new or updated content relevant to the patient's health and occupational conditions may be assigned to the patient.

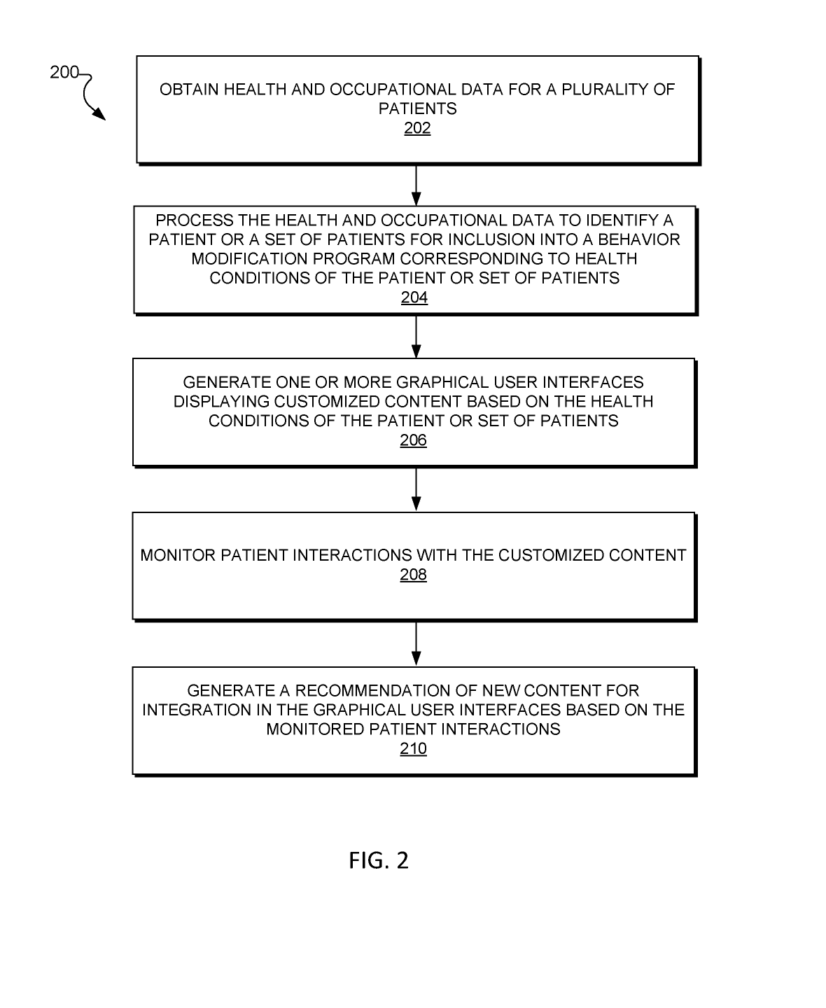

Referring now to FIG. 2 and with reference to FIG. 1, an illustrative process 200 for processing health and occupational data to identify patients, including workers, for inclusion in a behavioral modification program is provided. Referring initially to FIG. 2, process 200 begins with obtaining health and occupational data for a plurality of patients, each of which could be identified as a potential participant in a behavioral modification program. Referring to FIG. 1, health data may include, without limitation, claims data received by the server computing device 102 from the health data computing system 110 (or other health-related computing system, such as an electronic medical record system associated with a hospital, clinic, or other health care facility) and occupational data may include data received by the server computing device 102 from the occupational data computing system 111. As explained above, claims data may include data involving reimbursement for services rendered by a primary care doctor, a specialist, a hospital, a medical procedure, and/or the like. Occupational data may include, among other things, productivity, absenteeism, and presenteeism data associated with a particular worker and/or general information and data associated with a particular job description or occupational requirement.

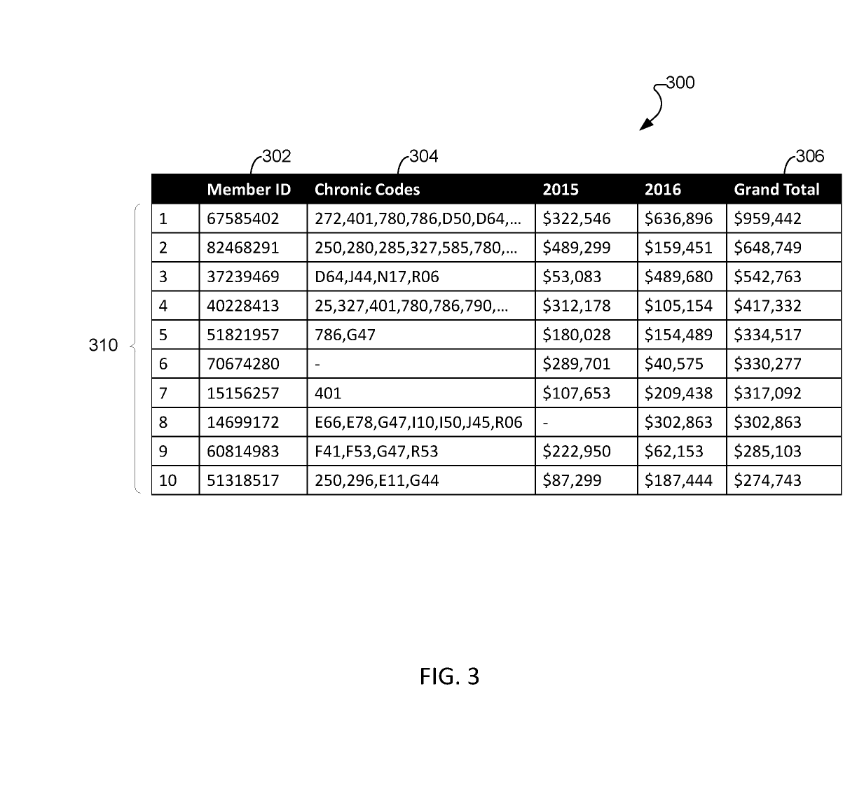

FIG. 3 illustrates an example of claim data in the form of a claims data source 300, according to one embodiment. As illustrated the data source 300 includes a table including a series of columns. In particular, the table includes a member ID column 302 that identifies a medical plan beneficiary. Additionally the table of the data source record 300A includes a chronic codes column 304 that indicates the number and type of chronic conditions which, when taken in combination, indicate whether an individual beneficiary is an appropriate candidate for a medical and behavioral treatment program. Finally, the table of the data source record 300 includes a Grand Total column 306 that defines the total cost to an employer for providing medical services to their health plan beneficiaries associated with such multiple chronic conditions. As further illustrated, the table of the billing record 300 includes one or more lines 310. A line may be a summary of multiple claims for services as aggregated for different individual beneficiaries. Each line includes a specific total amount value for the member ID column 302, chronic codes column 304, and Grand Total column 306.

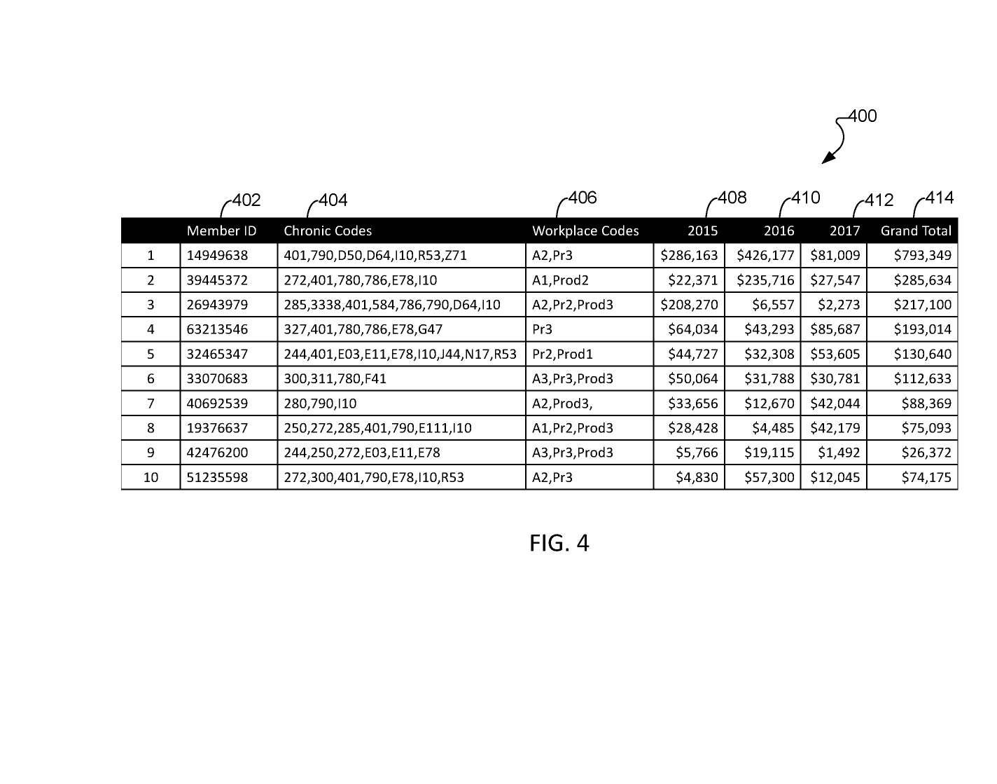

FIG. 4 illustrates an example of health and occupational data in the form of a combined data source 400, according to one embodiment. As illustrated the combined data source 400 includes a table including a series of columns. In particular, the table includes a member ID column 402 that includes a unique identifier for an employee or other workers of an organization. For each entry in the member ID column 402, the combined data source 400 further includes a chronic codes column 404 and a workplace codes column 406. Similar to the chronic codes column 304 of FIG. 3, the chronic codes column 404 of the combined data source 400 indicates the type of chronic conditions associated with a particular employee. The workplace codes column 406 includes a series of codes assigned to the employee reflecting the occupational performance of the employee. As illustrated in FIG. 4, in one implementation the occupational codes may include a letter or series of letters indicating a type of negative occupational data followed by a number indicating a severity or progression associated with the negative occupational data. For example, the codes in FIG. 4 include an “A”, “Pr”, and “Prod” indicating high absenteeism, high presenteeism, and low productivity, respectively. The codes further include a number from one to three that indicates whether the condition is periodic or improving (“1”), stable (“2”), or deteriorating (“3”). So, for example, the code “A1” may be used to indicate periodic or improving absenteeism, “Pr2” may be used to indicate stable or consistent presenteeism, and “Prod3” may be used to indicate deteriorating productivity or performance.

The combined data source 400 further includes columns 408, 410, 412 indicating total health care spend for each of the past three years and a grand total column 414 that sums the consumption over that same period. As discussed in the context of FIG. 3, such spend data may be calculated or otherwise obtained from claims data submitted by the members/employees.

The workplace codes column 406 may be populated in various ways. For example, in certain implementations, the workplace codes may be based on feedback provided by a supervisor or similar co-worker of the particular employee. Alternatively, the workplace codes may be generated based on an analysis of data stored within an occupational data computing system, such as the occupational data computing system 111 of FIG. 1. For example, the occupational data computing system may track various aspects of an employee's work including, without limitation, one or more of the employee's attendance, one or more productivity metrics associated with the employee's job, performance evaluations of the employee, and whether the employee is on a Performance Improvement Plan (PIP). This information may be used directly in determining a behavioral modification program for the employee or may be used to derive other codes or metrics, such as the “A”, “Pr”, and “Prod” codes discussed above.

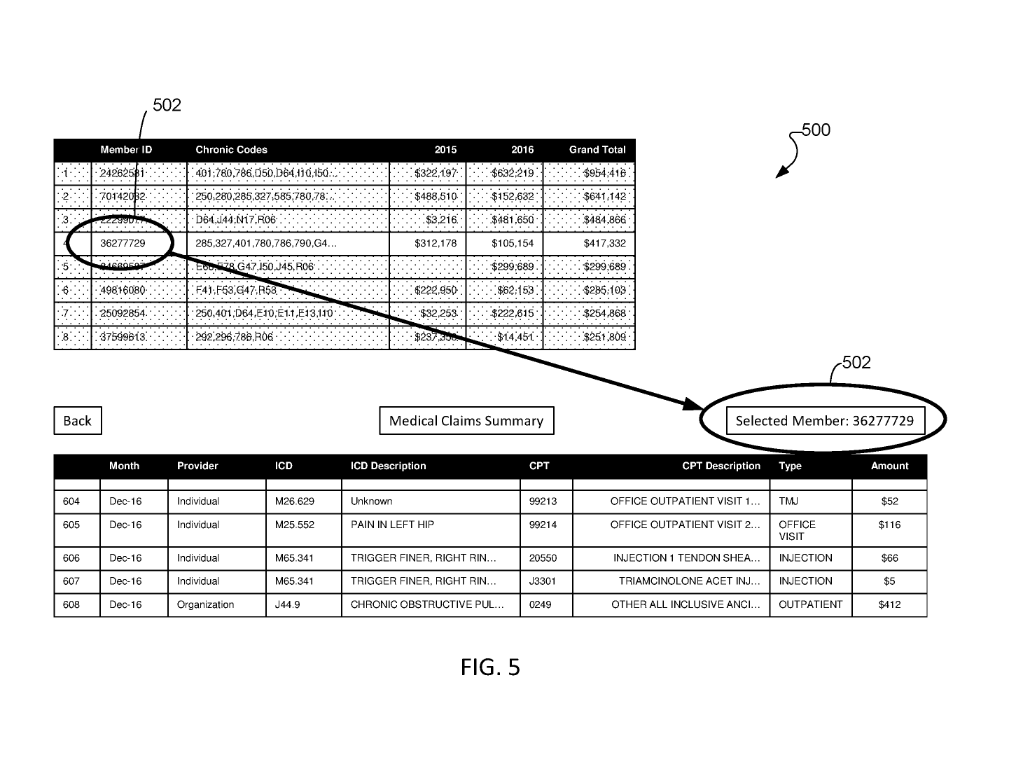

Referring back to FIG. 2, the obtained health and occupational data is processed to identify specific patients for inclusion into a behavioral modification program that is customized according to health conditions of that patient (operation 204). FIG. 5 illustrates an example of the processing of health data to identify those specific patients. As illustrated in the table of 500, patients are ranked by degree of utilization of healthcare services. Then, within the set of patients with high utilization of healthcare services, an algorithm is used to identify a specific pattern of chronic diseases which contribute to that high utilization. For example, in FIG. 5, the CPT codes signify the characteristics of the disease diagnoses which drive the patients' healthcare utilization. A unique collection of these CPT codes must be present in order for the patient to be a suitable candidate for the behavioral modification program. As a threshold in certain implementations, at least three distinctly different CPT code families may be required to be present for a patient to be considered qualified for the behavioral modification program, although other thresholds are contemplated. Also, each entry in the table 500 may connected to or otherwise linked to specific data for the particular member ID. For example, as illustrated in FIG. 5, the member ID 502 may be selected to “drill-down” into details for each of the claims associated with the patient having the member ID.

Identifying specific patients for inclusion in a behavioral modification program may also be based in whole or in part on occupational data, such as the occupational data collected from the occupational data computing system 111. For example, in certain implementations, employees may be ranked or otherwise ordered based on one or more of absenteeism, presenteeism, productivity, or any other metric tracked and stored within the occupational data computing system 111. Based on the ranking/ordering of the employees, a subset of the employees may be identified for participation in a behavioral modification program. For example, in certain implementations, the server computing device 102 may identify employees that fall within a certain percentage of all employees having one or more of the highest absenteeism, the highest presenteeism, or the lowest productivity. In other implementations, the server computing device 102 may identify each employee whose work-related data meets a predetermined threshold. In such implementations, an employee may be identified for participation in a behavioral modification program if the number of workdays missed (i.e., absenteeism) by the employee or the number of workdays during which the employee could only perform a limited set of tasks (i.e., presenteeism) exceed a certain number over a particular time period. For example, in one implementation, the server computing device 102 may be adapted to identify each employee having more than a predetermined number of absenteeism or presenteeism days over a month, quarter, half-year, year, or any other time period. In another implementation, the server computing device 102 may identify each employee having an average number of absenteeism or presenteeism days over a specified time period.

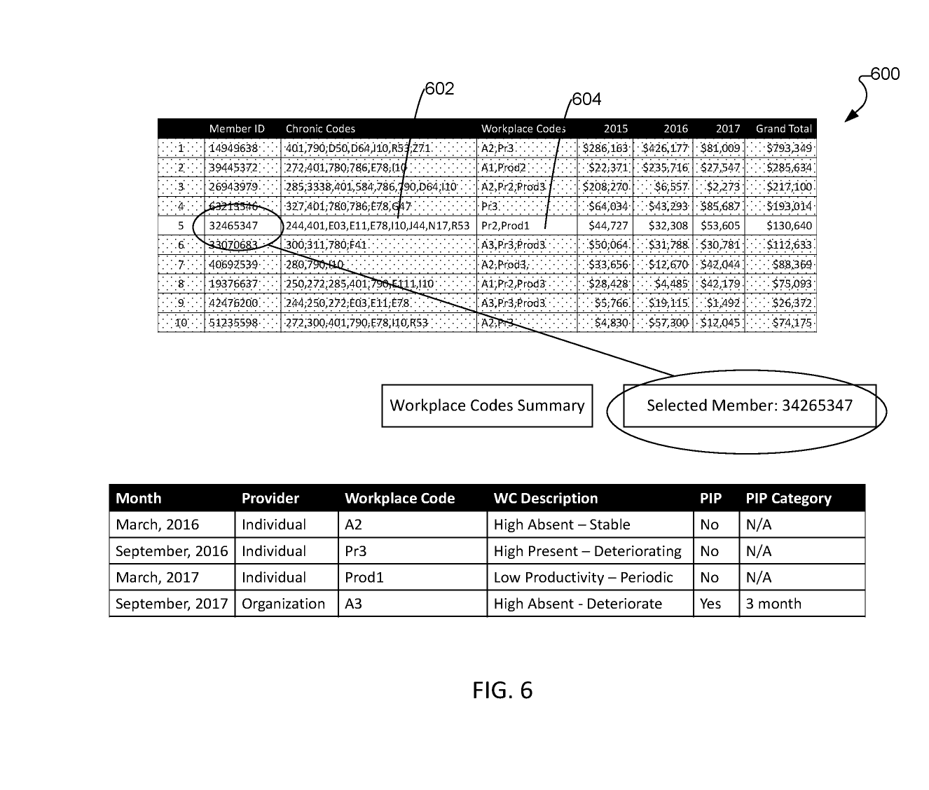

FIG. 6 illustrates an example of the processing of combined health and occupational data to identify specific patients for enrollment in a behavioral modification program. As illustrated in the table 600, patients may be ranked by degree of utilization of healthcare services. So, for example, the table 600 is ranked based on the grand total of healthcare services provided over the time period 2015-2017. Each entry in the table 600 may further include one or more code fields. For example, the table 600 includes each of a first field 602 for storing CPT or similar codes for identifying characteristics of diseases or other health issues corresponding to the patient's healthcare utilization and a second field 604 including a list of occupational codes corresponding to the patient's work performance or other work-related aspect.

FIG. 6 further illustrates a drill-down in which a more detailed listing of the occupational codes for a specific patient/employee is provided. As illustrated, the drill-down may provide further information regarding the occupational codes including, without limitation, the date particular codes were assigned, the entity responsible for assigning the codes, additional details regarding the code, and whether the patient/employee is enrolled in a corresponding performance improvement plan (PIP).

Identifying a particular patient/employee for enrollment in a behavioral modification program and the particular aspects and content included in a behavioral modification program in which a patient is enrolled is generally a function of the information stored within the table 600. For example, the decision to enroll a patient in a behavioral modification program may be based on, among other things, one or a combination of healthcare spend or similar financial metrics, the particular conditions suffered by the patient/employee, and trends in the patient/employees work performance. In one example implementation, the decision to enroll a patient/employee in a behavioral modification program may be based on a threshold for total economic impact (e.g., the total costs associated with healthcare spend and lost productivity associated with the worker), while the particular content included in the behavioral modification program may be dictated by the nature of the specific health conditions of the patient/employee.

In certain implementations, each of claims data and occupational data may be considered when identifying patients/employees for enrollment in a behavioral modification program. For example, in certain implementations, occupational data may be used to identify a subset of patient/employees that are then further evaluated based on claims data, or vice versa. By doing so, an employer may be able to readily identify, among other things, patients/employees with a combination of the highest health-related costs and the greatest amount of lost productivity such that the employer obtains the greatest value from the patient/employee's enrollment in a behavioral modification program.

The health data and the occupational data may also be assessed using a scoring system to identify candidates for behavioral modification programs. For example, points or similar values may be assigned to health and occupational data associated with a particular patient/employee and the patient/employee may be enrolled or offered to participate in a behavioral modification program if their score meets a predetermine threshold. In certain implementations, claim and work-related data may also be weighted to emphasize the importance of certain pieces of data. For example, in certain implementations, an employer may decide to assign greater weight to absenteeism days (in which an employee is completely absent from work) versus presenteeism days (in which an employee may still perform some limited tasks). Alternately, the fact that an employee is subject to a PIP may be a highly weighted piece of data, or a negative trend in productivity (output) metrics may be of high importance in determining whether an employee is a candidate for a treatment program.

Once a particular patient or a set of patients has been identified and enrolled in respective behavioral modification programs, custom content is provided to the identified patient or set of patients based on the conditions identified from the processed health and occupational data (operation 206). Referring to FIG. 1, in one specific example, the server computing device 102 may generate various graphical user interfaces that include interactive elements, such as survey questions, buttons, forms, activity logs, fields, streaming capabilities, selections, inputs, streams, images, etc., and/or charts, for displaying or otherwise presenting content to the patient that involves aspects of treatment or care corresponding to the set of conditions of the patient. Referring to FIG. 1, the patient may interact with the one or more communication devices 122₁, 122₂,-122_{N} to access the provided custom content.

Referring again to FIG. 2, in some embodiments, the system may monitor, in real-time, a patient's interactions with the provided custom content (operation 208). In one specific example, the server computing device 102 may employ the monitoring unit 108 to continuously monitor a patient's interactions and participation with the custom content provided in the generated graphical user interfaces. In one specific example, one or more parameters related to the behavioral modification program may be embedded in the graphical user interfaces displaying the custom content to patients and may be used to track patient interactions with the custom content. The parameter data may be tracked and continuously communicated to the server computing device 102. All of such interactions and/or parameter data may be stored, for example, in the database of server computing device 102.

Based on the patient interactions, the system may generate recommendations of new relevant content that may be automatically integrated into the platform and displayed at a client device for patient interaction (operation 210). Such recommended content may be identified or generated based on the monitoring performed by the server computing device 102.

For example, in one implementation, enrolled patients may be presented with one or more surveys in order to develop and customize a behavioral modification program that aligns more closely with a patient's specific health condition, work activities, and intrinsic motivation and goals. Such surveys may include, among other things, adaptability surveys (which provide a picture of how capable the patient is of handling and adapting to change, especially in his or her own behaviors), behavior and health surveys (which provide data on the patient's specific diseases and related health conditions), and activation surveys (which provide data regarding the patient's level of engagement with the behavioral modification program). Based on the results of such surveys and similar data collection, the server computing device 102 may customize or modify a particular patient's behavioral modification program in order to better suit the patient's behavior, motivations, and goals.

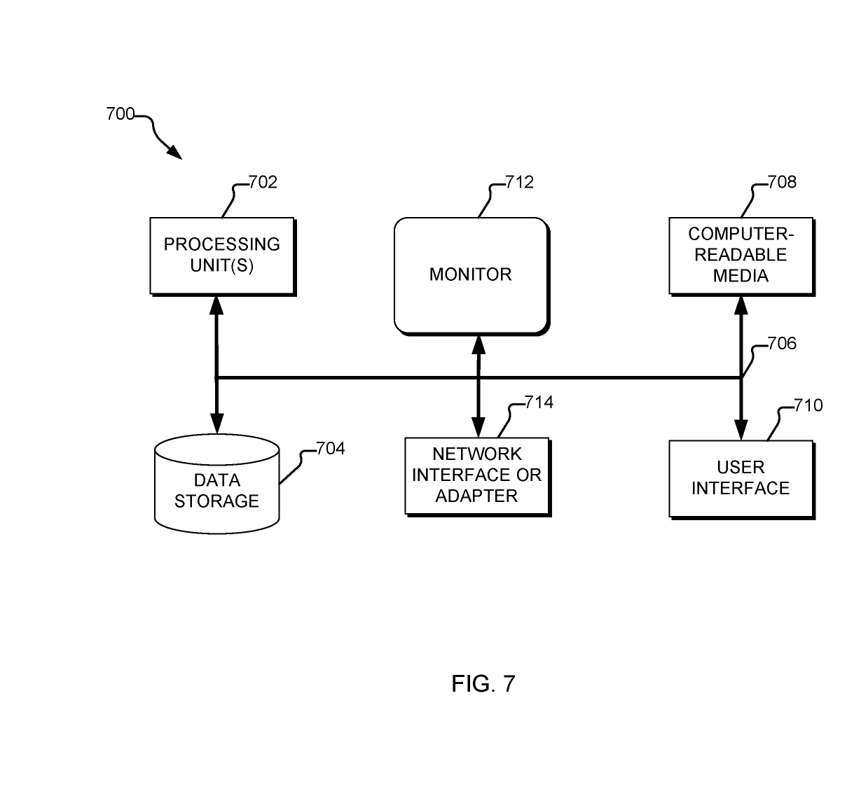

FIG. 7 illustrates an example of a suitable computing and networking environment 700 that may be used to implement various aspects of the present disclosure, including aspects described in FIGS. 1-2 such as the server computing device 102. As illustrated, the computing and networking environment 700 includes a general purpose computing device 700, although it is contemplated that the networking environment 700 may include one or more other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the computer 700 may include various hardware components, such as a processing unit 702, a data storage 704 (e.g., a system memory), and a system bus 706 that couples various system components of the computer 700 to the processing unit 702. The system bus 706 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 700 may further include a variety of computer-readable media 708 that includes removable/non-removable media and volatile/nonvolatile media, but excludes transitory propagated signals. Computer-readable media 708 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computer 700. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The data storage or system memory 704 includes computer storage media in the form of volatile/nonvolatile memory such as read only memory (ROM) and random access memory (RAM). A basic input/output system (BIOS), containing the basic routines that help to transfer information between elements within the computer 700 (e.g., during start-up) is typically stored in ROM. RAM typically contains data and/or program modules that are immediately accessible to and/or presently being operated on by processing unit 702. For example, in one embodiment, data storage 704 holds an operating system, application programs, and other program modules and program data.

Data storage 704 may also include other removable/non-removable, volatile/nonvolatile computer storage media. For example, data storage 704 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media, described above and illustrated in FIG. 7, provide storage of computer-readable instructions, data structures, program modules and other data for the computer 700.

A user may enter commands and information through a user interface 710 or other input devices such as a tablet, electronic digitizer, a microphone, keyboard, and/or pointing device, commonly referred to as mouse, trackball or touch pad. Other input devices may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices are often connected to the processing unit 702 through a user interface 710 that is coupled to the system bus 706, but may be connected by other interface and bus structures, such as a parallel port, game port or a universal serial bus (USB). A monitor 712 or other type of display device is also connected to the system bus 706 via an interface, such as a video interface. The monitor 712 may also be integrated with a touch-screen panel or the like.

The computer 700 may operate in a networked or cloud-computing environment using logical connections of a network interface or adapter 714 to one or more remote devices, such as a remote computer. The remote computer may be a personal computer, a server, a router, a network PC, a peer device or other common network node, and typically includes many or all of the elements described above relative to the computer 700. The logical connections depicted in FIG. 7 include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computer 700 may be connected to a public and/or private network through the network interface or adapter 714. In such embodiments, a modem or other means for establishing communications over the network is connected to the system bus 706 via the network interface or adapter 714 or other appropriate mechanism. A wireless networking component including an interface and antenna may be coupled through a suitable device such as an access point or peer computer to a network. In a networked environment, program modules depicted relative to the computer 700, or portions thereof, may be stored in the remote memory storage device.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. A system comprising:
a processing device; and
a memory containing one or more instructions that, when executed by the processing device, cause the processing device to:
obtain health data from one or more first data sources, the health data corresponding to a plurality of patients;
obtain occupational data from one or more second data sources, the occupational data corresponding to the plurality of patients;
process the health data and the occupational data to identify at least one patient of the plurality of patients for inclusion in a behavioral modification program for at least one health condition corresponding to the patient;
generate one or more graphical user interfaces of the behavioral modification program for display at a client device, the one or more graphical user interfaces displaying first content, wherein the first content is associated with the at least one health condition;
monitor user interactions with the first content; and
integrate second content into the graphical user interfaces based on the monitored user interactions with the first content.

2. The system of claim 1, wherein the processing device is further configured to:
monitor user interactions with the second content; and
integrate third content into the graphical user-interfaces based on the user interactions with the second content.

3. The system of claim 1, wherein the health data includes claims data describing medical services provided to the plurality of patients.

4. The system of claim 3, wherein the claims data includes a table comprising a plurality of columns including at least one of an employee column, a chronic codes column, and a grand total column.

5. The system of claim 1, wherein the computing device is further configured to identify health data element deficiencies and automatically correct health data element deficiencies.

6. The system of claim 1, wherein the first content is multimedia content including at least one of audio, video, and image content.

7. The system of claim 1, wherein the occupational data includes at least one of patient absenteeism, patient presenteeism, and patient productivity.

8. A method comprising:
obtaining, using a computing device, health data from one or more first data sources, the health data corresponding to a plurality of patients;
obtaining, using a computing device, occupational data from one or more second data sources, the occupational data corresponding to the plurality of patients;
processing, using the computing device, the health data and the occupational data to identify at least one patient of the plurality of patients for inclusion in a behavioral modification program for at least one health condition corresponding to the patient;
generating, using the computing device, one or more graphical user interfaces of the behavioral modification program for display at a client device, the one or more graphical user interfaces displaying first content, wherein the first content is associated with the at least one chronic condition;
monitoring, using the computing device, user interactions with the first content; and
integrating, using the computing device, second content into the graphical user interfaces based on the monitored user interactions with the first content.

9. The method of claim 8, further comprising:
monitoring, using the computing device, user interactions with the second content; and
integrating, using the computing device, third content into the graphical user interfaces based on the user interactions of the second content.

10. The method of claim 8, wherein the health data includes claims data describing medical services provided to the plurality of patients.

11. The method of claim 10, wherein the claims data includes a table comprising a plurality of columns including at least one of an employee column, a chronic codes column, and a grand total column.

12. The method of claim 8, further comprising:
identifying, using the computing device, one or more health data element deficiencies; and
correcting, using the computing device, the one or more health data element deficiencies.

13. The method of claim 8, wherein the first content is multimedia content including at least one of audio, video, and image content.

14. A non-transitory computer readable medium encoded with instructions executable by a computing device such that, when executed by the computing device, the instructions cause the computing device to:
obtain health data from one or more first data sources, the health data corresponding to a plurality of patients;
obtain occupational data from one or more second data sources, the occupational data corresponding to the plurality of patients;
process the health data and the occupational data to identify at least one patient of the plurality of patients for inclusion in a behavioral modification program and at least one health condition corresponding to the patient;
generate one or more graphical user-interfaces of the behavioral modification program for display at a client device, the one or more graphical user interfaces displaying first content, wherein the first content is associated with the at least one chronic condition;
monitor user interactions with the first content; and
integrate second content into the graphical user interfaces based on the monitored user interactions with the first content.

15. The non-transitory computer readable medium of claim 14, wherein the instructions further cause the computing device to:
monitor user interactions with the second content; and
integrate third content into the graphical user interfaces based on the user interactions of the second content.

16. The non-transitory computer readable medium of claim 14, wherein the health data is claims data describing medical services provided to the plurality of patients.

17. The non-transitory computer readable medium of claim 16, wherein the claims data includes a table comprising a plurality of columns including at least one of an employee column, a chronic codes column, and a grand total column.

18. The non-transitory computer readable medium of claim 14, wherein the instructions further cause the computing device to identify health data element deficiencies and to automatically correct the claim data element deficiencies.

19. The non-transitory computer readable medium of claim 14, wherein the first content is multimedia content including at least one of audio, video, and image content.

20. The non-transitory computer readable medium of claim 14, wherein the occupational data includes at least one of patient absenteeism, patient presenteeism, and patient productivity.
